(19) United States
(12) Patent Application Publication
(10) Pub. No.: US 2019/0358627 A1
(43) Pub. Date: Nov. 28, 2019

(54) MAGNETOPHORESIS BIOCHIP

(71) Applicant: ZITRONICS INC., Siheung-Si Gyeonggi-do (KR)

(72) Inventors: Jihwang Park, Goyang-Si Gyeonggi-do (KR); Eunseok Nam, Goyang-Si Gyeonggi-do (KR)

(73) Assignee: ZITRONICS INC., Siheung-Si Gyeonggi-do (KR)

(21) Appl. No.: 16/456,473

(22) Filed: Jun. 28, 2019

Related U.S. Application Data
(63) Continuation of application No. PCT/KR2018/006331, filed on Jun. 1, 2018.

(30) Foreign Application Priority Data
Jun. 2, 2017 (KR) 10-2017-0068974

(51) Int. Cl. B01L 3/00; G01N 33/543; B03C 1/01; B03C 1/28; B03C 1/033

(52) U.S. Cl. CPC B01L 3/50273; G01N 33/54326; B01L 3/502761; B03C 1/01; B03C 1/288; B03C 1/0332; B03C 2201/22; B01L 2300/0819; B01L 2300/0861; B01L 2400/043; B03C 2201/18; B03C 2201/26; B01L 2200/0652

(57) ABSTRACT

A magnetophoresis biochip is provided. The magnetophoresis biochip includes a magnetic force providing unit including a 1-1 surface having magnetic force of a 1-1 pole, and a 2-1 surface which is spaced apart from the 1-1 surface and faces each other and has magnetic force of a 2-1 pole that is opposite to the 1-1 pole, a magnetic force shield including a first magnetic force shield blocking magnetic force of a 1-2 pole of opposite side of surface facing the 2-1 surface in the 1-1 surface, and a second magnetic force shield blocking magnetic force of a 2-2 pole of opposite side of surface facing the 1-1 surface in the 2-1 surface, and a biochip including 3 or more injection channels, a mixing channel and 3 or more separation channels, which are located between the 1-1 surface and the 2-1 surface and extended in one direction and arranged in sequence.

### CROSS-REFERENCE TO RELATED APPLICATIONS

This application is a continuation of International Patent Application No. PCT/KR2018/006331 filed on Jun. 1, 2018, which claims priority to Korean Patent Application No. 10-2017-0068974 filed on Jun. 2, 2017, the entire contents of which are herein incorporated by reference.

### TECHNICAL FIELD

The present invention relates to a magnetophoresis biochip.

### BACKGROUND ART

In recent years, a device or a method for detecting and quantifying biomolecules such as metabolism products or biomarkers of diseases for new drug development or medical diagnosis with high sensitivity has been demanded with the development of medical technologies. As a method for detecting such biomolecules, a binding assay method is widely used, and immunoassay, DNA hybridization and receptor-based assay belong to it. However, since it is not possible to directly observe binding in case of biomolecules, the presence of biomolecules targeted is confirmed by using a target material in the binding assay method. This target material includes radioactive materials, fluorescent materials, enzyme labels, magnetic particles, etc.

Among them, the magnetic particles attract much attention as a labeling material of binding assay, since it can easily control the movement of magnetic particles due to magnetic property, and have high biocompatibility, and have advantages such as high sensitivity. As a method using the magnetic particles, magnetic particles having a probe capable of combining with a target biomolecule on the surface are injected in a sample solution and the target biomolecule is captured (combined), and the magnetic particles are separated from the sample solution again, thereby selecting and extracting only the target biomolecule. On this wise, a method for separating a target biomolecule by using magnetic particles (bead based separation) is widely used for separating cells, proteins, nucleic acids, or other biomolecules, etc. For example, U.S. Pat. No. 6,893,881 discloses a method for separating a specific target cell using an antibody-coated paramagnetic bead. Like this, when a magnetic field is applied to molecules having a magnetic property, magnetic particles can be detected and further quantified by using that the route of magnetic particles is changed by the magnetic field, and this method is called magnetophoresis.

In recent years, according to the rapidly increasing bio information, rapid processing is difficult with the conventional laboratory analysis system, and biological detection systems for identification of life phenomena, and new drug development and diagnosis have been developed as a form of micro comprehensive analysis system and lab-on-a-chip for analyzing a sample accurately and conveniently in a short time with a smaller quantity on the basis of microfluidics. Thus, a biochip capable of analyzing a sample accurately and conveniently in a short time with a smaller quantity while using magnetic particles having advantages of easy motion control, high biocompatibility and high sensitivity has been required.

### SUMMARY

Accordingly, a problem to be solved by the present invention is to provide a magnetophoresis biochip capable of more precisely separating a target biomolecule targeted from a biomaterial more effectively.

In addition, it is to provide a magnetophoresis biochip capable of analyzing a sample accurately and conveniently in a short time with a small quantity in a smaller size.

Moreover, it is to provide a magnetophoresis biochip capable of solving problems occurred in a use of magnetic particle as a target material. In other words, it is to provide a magnetophoresis biochip capable of preventing the function of biochip from deteriorating, by accumulating the magnetic particle on a channel of specific location by using the magnetic particle as a target material.

Furthermore, it is to provide a magnetophoresis biochip capable of separating multiple target biomolecules more effectively even in a single series of processes.

The problems of the present invention are not limited to the above-mentioned technical problems, and other technical problems which are not mentioned can be clearly appreciated by those skilled in the art from the following description.

The magnetophoresis biochip according to one example of the present invention to solve the problem is characterized by comprising a magnetic force providing unit including a 1-1 surface having magnetic force of a 1-1 pole, and a 2-1 surface which is spaced apart from the 1-1 surface and faces each other and has magnetic force of a 2-1 pole that is opposite to the 1-1 pole, a magnetic force shield including a first magnetic force shield blocking magnetic force of a 1-2 pole of opposite side of surface facing the 2-1 surface in the 1-1 surface, and a second magnetic force shield blocking magnetic force of a 2-2 pole of opposite side of surface facing the 1-1 surface in the 2-1 surface, and a biochip including 3 or more injection channels, a mixing channel and 3 or more separation channels, which are located between the 1-1 surface and the 2-1 surface and extended in one direction and arranged in sequence, wherein an angle formed by connecting a virtual extended surface of the 1-1 surface and the 2-1 surface is in the range of more than 0° to less than 90°.

The first magnetic force shield may be formed by that the 1-1 pole of the magnetic force providing unit is extended in the direction opposite to the 2-1 surface in the 1-1 surface, and the second magnetic force shield may be formed by that the 2-1 pole of the magnetic force providing unit is extended in the direction opposite to the 1-1 surface in the 2-1 surface, and the first magnetic force shield may have a ratio of a width of the 1-1 surface and an extended height of the 1-1 pole of 1:3 or more to 1:100 or less, and the second magnetic force shield may have a ratio of a width of the 2-1 surface and an extended height of the 2-1 pole of 1:3 or more to 1:100 or less.

The magnetic force shield is formed by that the 1-1 pole of the magnetic force providing unit is extended, and it is formed by that the 2-1 pole is extended, and the end of extended part of the 1-1 pole and the end of extended part of the 2-1 pole may in contact with each other.

The first magnetic force shield may be arranged as spaced apart from the 1-2 surface which is the opposite surface of the 1-1 surface facing the 2-1 surface, and is formed with the same pole as the 1-1 pole, and the second magnetic force shield may be arranged as spaced apart from the 2-2 surface which is the opposite surface of the 2-1 surface facing the 1-1 surface, and is formed with the same pole as the 2-1 pole.

The first magnetic force shield may prevent the magnetic force by the 1-2 pole from overlapping with the magnetic field formed between the 1-1 surface and the 2-1 surface, by being contact with or spaced apart from the 1-2 surface which is the opposite surface to the 1-1 surface facing the 2-1 surface to induce the magnetic force of the 1-2 pole in a specific direction, and the second magnetic force shield may prevent the magnetic force by the 2-2 pole from overlapping with the magnetic field formed between the 1-1 surface and the 2-1 surface, by being contact with or spaced apart from the 2-2 surface which is the opposite surface to the 2-1 surface facing the 1-1 surface to induce the magnetic force of the 2-2 pole in a specific direction.

An angle formed by connecting a virtual extended surface of the 1-1 surface and the 2-1 surface may in the range of more than 0° to 50° or less.

At least a part of the 1-1 surface or the 2-1 surface may include a plane or a curved surface.

The ratio of the length of width of the 1-1 surface or the 2-1 surface and the closest distance between the 1-1 surface and the 2-1 surface may be in the range of 30:1 to 1:1.

In the biochip, a magnetic substance, in which a probe for inducing an immunobinding with a biomolecule on a surface is formed, may be injected to at least one or more of the 3 or more injection channels, and a biomaterial containing a target biomolecule to be separated may be injected to the other 1 or more, and in the mixing channel, the biomaterial and the magnetic substance may be mixed with each other, and the target biomolecule and the magnetic substance may be combined by immunobinding to form a conjugate, and the conjugate may pass through at least one or more of the 3 or more of separation channels, and the biomaterial may pass through the other 1 or more.

The biochip may comprise 4 or more of injection channels, a mixing channel and 4 or more of separation channels, and the magnetic substance may comprise a first magnetic substance and a second magnetic substance which have a different size from each other or characteristic of magnetization, and the first magnetic substance and the second magnetic substance form a probe for inducing an immunobinding with a different biomolecule each other respectively, and in the mixing channel, the first magnetic substance and the second magnetic substance may be combined with each target biomolecule to form a first conjugate and a second conjugate, and in the separation channel, the first conjugate and the second conjugate may pass through a respectively different channel.

Other specific details of examples are included in detailed description and drawings.

There are at least the following effects by examples of the present invention.

According to the present invention, a target biomolecule targeted can be more precisely separated from a biomaterial more effectively.

In addition, a sample can be analyzed accurately and conveniently in a short time with a small quantity in a smaller size.

Moreover, it can be prevented that the function of separating a biomaterial of biochip is deteriorated, by accumulating a magnetic particle on a channel of specific location, which is a problem possible to be occurred for using a magnetic particle as a target material.

Furthermore, multiple target biomolecules can be more effectively separated even in a single series of processes.

The effects according to the present invention are not limited by the contents exemplified above, and more various effects are included in the present specification.

### DESCRIPTION OF DRAWINGS

FIG. 1 is a perspective view showing a magnetophoresis biochip of one example of the present invention schematically.

FIG. 2 is a sectional view of magnetophoresis biochip according to FIG. 1.

FIG. 3 is a sectional view magnifying A part in the magnetophoresis biochip according to FIG. 1.

FIG. 4 is a plane view showing the biochip part in the magnetophoresis biochip according to FIG. 1 schematically.

FIG. 5 is the result of interpretation of magnetic field according to the simulation of comparative example in case that the angle formed by connecting the virtual extended surface of the 1-1 surface and the 2-1 surface is 0° when there is no magnetic field shield.

FIG. 6 is the result of interpretation of magnetic field according to the simulation of comparative example in case that the angle formed by connecting the virtual extended surface of the 1-1 surface and the 2-1 surface is 10° when there is no magnetic field shield.

FIG. 7 is the result of interpretation of magnetic field according to the simulation of comparative example in case that the angle formed by connecting the virtual extended surface of the 1-1 surface and the 2-1 surface is 20° when there is no magnetic field shield.

FIG. 8 is the result of interpretation of magnetic field according to the simulation of comparative example in case that the angle formed by connecting the virtual extended surface of the 1-1 surface and the 2-1 surface is 30° when there is no magnetic field shield.

FIG. 9 is the result of interpretation of magnetic field according to the simulation of comparative example in case that the angle formed by connecting the virtual extended surface of the 1-1 surface and the 2-1 surface is 40° when there is no magnetic field shield.

FIG. 10 is the result of interpretation of magnetic field according to the simulation of comparative example in case that the angle formed by connecting the virtual extended surface of the 1-1 surface and the 2-1 surface is 0° when there is the magnetic field shield.

FIG. 11 is the result of interpretation of magnetic field according to the simulation of comparative example in case that the angle formed by connecting the virtual extended surface of the 1-1 surface and the 2-1 surface is 10° when there is the magnetic field shield.

FIG. 12 is the result of interpretation of magnetic field according to the simulation of comparative example in case that the angle formed by connecting the virtual extended surface of the 1-1 surface and the 2-1 surface is 20° when there is the magnetic field shield.

FIG. 13 is the result of interpretation of magnetic field according to the simulation of comparative example in case that the angle formed by connecting the virtual extended surface of the 1-1 surface and the 2-1 surface is 30° when there is the magnetic field shield.

FIG. 14 is the result of interpretation of magnetic field according to the simulation of comparative example in case that the angle formed by connecting the virtual extended surface of the 1-1 surface and the 2-1 surface is 40° when there is the magnetic field shield.

FIG. 15 is the result of interpretation of magnetic field according to the simulation of comparative example in case that the angle formed by connecting the virtual extended surface of the 1-1 surface and the 2-1 surface is 50° when there is the magnetic field shield.

FIG. 16 is the result of magnetic force showed by simulating with an angle formed by connecting the virtual extended surface of the 1-1 surface and the 2-1 surface of the magnetophoresis biochip according to the example of the present invention.

FIG. 17 is the result of magnetic force showed by simulating with an angle formed by connecting the virtual extended surface of the 1-1 surface and the 2-1 surface of the magnetophoresis biochip according to the example of the present invention.

FIG. 18 is the result of magnetic force showed by simulating with an angle formed by connecting the virtual extended surface of the 1-1 surface and the 2-1 surface of the magnetophoresis biochip according to the example of the present invention.

FIG. 19 is the result of magnetic force showed by simulating with an angle formed by connecting the virtual extended surface of the 1-1 surface and the 2-1 surface of the magnetophoresis biochip according to the example of the present invention.

FIG. 20 is the result of magnetic force showed by simulating with an angle formed by connecting the virtual extended surface of the 1-1 surface and the 2-1 surface of the magnetophoresis biochip according to the example of the present invention.

FIG. 21 is the result of magnetic force showed in a small deviation range to a certain value of the size of magnetic force by simulating in case that the angle formed by connecting the virtual extended surface of the 1-1 surface and the 2-1 surface is 35° when the closest distance between the 1-1 surface and the 2-2 surface of the magnetophoresis biochip according to the example of the present invention is 3 mm.

FIG. 22 is the result of magnetic force showed in a small deviation range to a certain value of the size of magnetic force by simulating in case that the angle formed by connecting the virtual extended surface of the 1-1 surface and the 2-1 surface is 28° when the closest distance between the 1-1 surface and the 2-2 surface of the magnetophoresis biochip according to the example of the present invention is 2 mm.

FIG. 23 is a sectional view showing the magnetophoresis biochip of another example of the present invention schematically.

FIG. 24 is a sectional view showing the magnetophoresis biochip of other example of the present invention schematically.

FIG. 25 is a sectional view showing the magnetophoresis biochip of other example of the present invention schematically.

FIG. 26 is a sectional view showing the magnetophoresis biochip of other example of the present invention schematically.

### DETAILED DESCRIPTION

The advantages and features of the present invention, and methods for achieving them will become apparent with reference to the examples described in detail below with accompanying drawings. However, the present invention is not limited to the examples described below, but may be embodied in various different forms, and the present examples are provided only for completing the disclosure of the present invention and completely notifying the scope of the invention to those skilled in the art, and the present invention is only defined by the scope of claims. Same references refer to same components throughout the specification. The size of layers and regions and the relative size in the drawings may be exaggerated for clarity of illustration.

The spatially relative terms, “below”, “beneath”, “lower”, “above”, “upper”, etc. can be used to easily describe the correlation of one device or component with other device or components.

Although first, second, etc. are used to describe various components, it goes without saying that these components are not limited by these terms. These terms are used only for distinguish one component from other component. Therefore, it goes without saying that the first component mentioned below may be the second component within the technical spirit of the present invention.

Hereinafter, examples of the present invention will be described with reference to drawings.

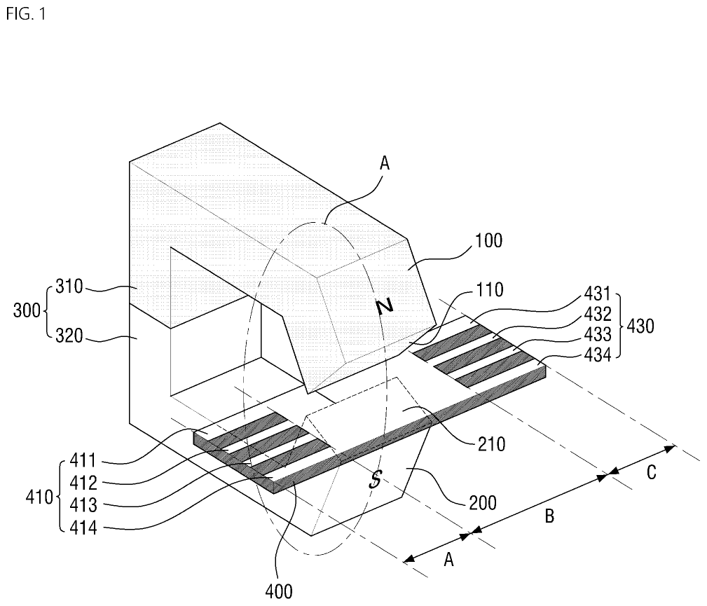

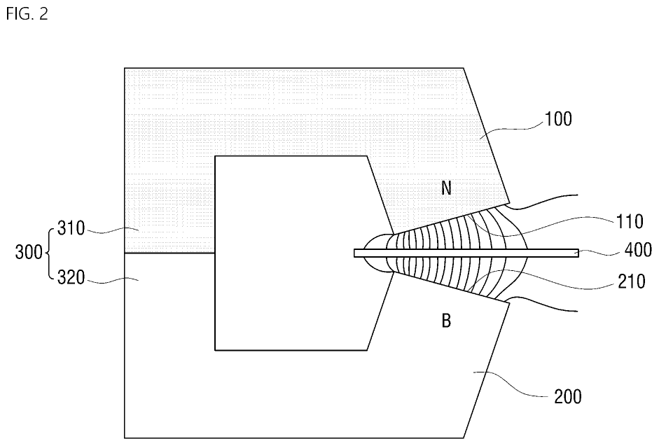

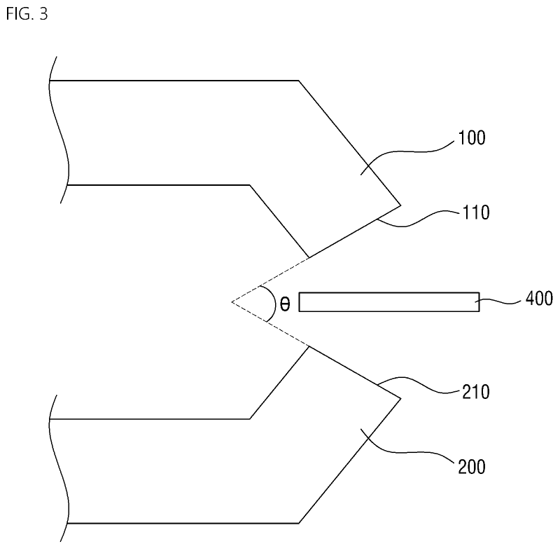

FIG. 1 shows a perspective view according to one example of magnetophoresis biochip of the present invention, and FIG. 2 shows a sectional view of FIG. 1, and FIG. 3 shows a sectional view magnifying A part in the perspective view of FIG. 1. Hereinafter, the magnetophoresis biochip according to one example of the present invention will be described with reference to FIG. 1 to FIG. 3.

Referring to FIG. 1 to FIG. 3, the magnetophoresis biochip comprises a magnetic force providing unit (100, 200) including a 1-1 surface (110) having magnetic force of a 1-1 pole (N), and a 2-1 surface (210) which is spaced apart from the 1-1 surface (110) and faces each other and has magnetic force of a 2-1 pole (S) that is opposite to the 1-1 pole (N), a magnetic force shield (300) including a first magnetic force shield (310) blocking magnetic force of a 1-2 pole of opposite side of surface facing the 2-1 surface (210) in the 1-1 surface (110), and a second magnetic force shield (320) blocking magnetic force of a 2-2 pole of opposite side of surface facing the 1-1 surface (110) in the 2-1 surface (210), and a biochip (400) including 3 or more injection channels (411, 412, 413, 414), a mixing channel (420) and 3 or more separation channels (431, 432, 433, 434), which are located between the 1-1 surface (110) and the 2-1 surface (210) and extended in one direction and arranged in sequence, in which an angle formed by connecting a virtual extended surface of the 1-1 surface (110) and the 2-1 surface (210) is in the range of more than 0° to less than 90°.

In the magnetic providing unit (100, 200), the 1-1 surface (110) and the 2-1 surface (210), which are spaced apart and face have opposite magnetic forces each other. In other words, in FIG. 1, it is represented that the 1-1 pole is N pole and the 2-1 pole is S pole, but it is not limited thereto, and the N pole and S pole may be reversed. The 1-2 pole may have the magnetic pole opposite to the 1-1 pole (N), and the 2-2 pole may have the magnetic pole opposite to 2-1 pole (S). In other words, the 1-2 pole may have the same magnetic pole as the 2-1 pole (S), and the 2-2 pole may have the same magnetic pole as the 1-1 pole (N). On the other hand, as will be described below, the 1-2 pole may be connected to the part forming the 2-2 pole, and in this case, the 1-2 pole may be located in the end formed by extending the 2-1 pole (S) and the 2-2 pole may be located in the end formed by extending the 1-1 pole (N).

In addition, the magnetic field providing unit (100, 200) is represented as a permanent magnet in the drawings, but not limited thereto, and may be composed of electromagnet. In other words, the magnetic field providing unit (100, 200) may have a magnetic force that is opposite in magnetic force of the surface facing each other. The magnetic field providing unit (100, 200) is under the effect of magnetic field only for the close range of the surface facing each other by the magnetic force shield (300) described below, and it is substantially hardly affected by other magnetic poles except for the 1-1 pole (N) and the 2-1 pole (S) facing each other, and thereby the range capable of separating target biomolecules in the biochip can be broaden. On the other hand, this will be discussed in more detail in the followings through the simulation.

The magnetic force shield (300) comprises a first magnetic force shield (310) and a second magnetic force shield (320). More specifically, the first magnetic force shield (310) may block the magnetic force of opposite side of the surface facing the 2-1 surface (210) in the 1-1 surface (110) or minimize the level of affecting the 1-1 surface (110) and the 2-1 surface (210). In other words, the first magnetic force shield (310) removes or offsets a magnetic force different from the 1-1 pole (N) in the part connected to the 1-1 surface (110) having the 1-1 pole (N), or prevents or inhibits the effect of magnetic force of other parts on the biochip (400).

Likewise, the second magnetic field shield (320) may block the magnetic force of opposite side of the surface facing the 1-1 surface (110) in the 2-1 surface (210) or minimize the level of affecting the 1-1 surface (110) and the 2-1 surface (210). In other words, it removes or offsets a magnetic force different from the 2-1 pole (S) as located in the direction away from the 1-1 surface (110) in the extended part of the 2-1 surface (210), or prevents or inhibits the effect of magnetic force of other parts on the biochip (400).

To explain the magnetic force shield (300) more specifically, when there is a magnetic force gradient between the 1-1 pole (N) and the 2-1 pole (S) by the magnetic force providing unit (100, 200), the magnetic substance passing the inside of biochip (400) captures a target biomolecule and makes it moved and separated, and then other magnetic forces present in the outside other than the 1-1 pole (N) and the 2-1 pole (S) may cause interference, and the magnetic force shield (300) removes or minimizes such an interference, thereby broadening the available range of biochip (400) and making usable efficiently in various ways. The magnetic force shield (330) may be in various forms, and this will be described in more detail below with reference to drawings.

The magnetic force shield (300) may be formed by extending the 1-1 pole (N) of the magnetic force providing unit, and be formed by extending the 2-1 pole (S), and the end of extended part of the 1-1 pole (N) and the end of extended part of 2-1 pole (S) (310, 320) may be in contact with each other. In other words, as FIG. 1 and FIG. 2, the magnetic force shield (300) may be formed by that the 1-1 pole (N) and the 2-1 pole (S) are extended in the opposite direction from the direction facing the 1-1 surface (110) and the 2-1 surface (210) and bent each other and connected to each other in one place in a shape like a horseshoe. In this case, the 1-2 pole and the 2-2 pole of magnetic poles different each other may be in contact with each other. By this, other magnetic forces other than the 1-1 pole (N) and the 2-1 pole (S) are offset in the connected part, thereby excluding the influence of other magnetic forces other than the 1-1 pole (N) and the 2-1 pole (S) or minimizing their influence.

On the other hand, the biochip (400) is located between the 1-1 surface (110) and the 2-1 surface (210) and extended in one direction. The biochip (400) may be located by being all included in the virtual space between the 1-1 surface (110) and the 2-1 surface (210), or located by being partially overlapped. The biochip (400) is not contact to the 1-1 surface (110) and the 2-1 surface (210), and may be arranged as spaced apart partially. In addition, assuming a virtual three-dimensional figure constituting the space of the 1-1 surface (110) and the 2-1 surface (210), it may be located at the center of gravity of such a three-dimensional figure. However, it is not limited thereto.

One direction in which the biochip (400) is extended may be different by magnetic forces graded by the 1-1 pole (110) and the 2-1 pole (120), but if the direction from the 1-1 pole (N) to the 2-1 pole (120) is a vertical direction, the virtual direction passing between the 1-1 surface (110) and the 2-1 surface (210) by starting from the part connecting the virtual extended surface of the 1-1 surface (110) and the 2-1 surface (210) can be defined as a vertical direction on the horizontal plane. In other words, referring to FIG. 1 and FIG. 2, the part connecting the virtual extended surface of the 1-1 surface (110) and the 2-1 surface (210) of the magnetic force providing unit (100, 200) has θ angle, and the direction passing the core between the 1-1 surface (110) and the 2-1 surface (210) from the part constituting θ angle again can be defined as the second direction, and the vertical direction on the horizontal plane for the second direction may be one direction. Referring to FIG. 3, when viewing the drawing from the front, the direction of penetrating the ground in the viewing area can be defined as one direction.

The biochip (400) may be a hexahedron having a thin thickness comprising a planar shape when viewing from the outside, but not limited thereto, and it may be appropriately modified by those skilled in the art as required.

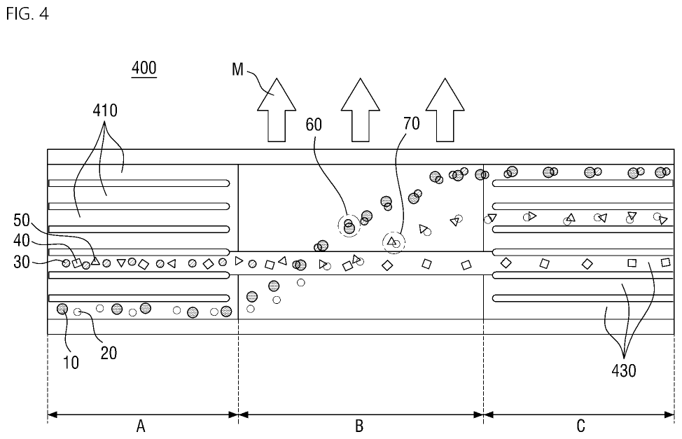

FIG. 4 shows a horizontal sectional view schematically showing the magnified biochip (400) part in the magnetphoresis biochip of FIG. 1. In other words, if FIG. 2 and FIG. 3 are vertical sectional views, FIG. 4 is appreciated as a sectional view taken along a horizontal section.

Referring to FIG. 4, the biochip (400) can be divided into largely three areas. A first region (A) in which injection channels (410) are located, a second region (B) in which the mixing channel (420) is located, and a third region (C) in which the separation channels (430) are located are comprised, and the first region (A), the second region (B) and the third region (C) may be formed sequentially. However, it is not limited thereto, and some other components may be present between the first region (A) to the third region (C).

The biochip (400) comprises 3 or more of injection channels (410) arranged in order in the first region (A), and comprises the mixing channel (420) in the second region (B), and comprises 3 or more of separation channels (430) arranged in order in the third region (C).

More specifically, 3 or more of injection channels (410) are arranged in order in the first region (A). In FIG. 4, 7 injection channels (410) are arranged, and referring back to FIG. 1, 4 injection channels (411, 412, 413, 414) are arranged in order. The size of injection channel (410), that is its width may be in the unit of minute micro (pm) or unit of nanometer (nm), but not limited thereto, and the size may be appropriately controlled by those skilled in the art as required. In other words, the injection channel in which a sample consisting of biomaterials (30, 40, 50) including target biomolecules (30, 50) is injected may be bigger than other injection channels, and the reverse may be possible as needed.

The mixing channel (420) is equipped in the second region (B), and the mixing channel (420) is the part performing the mixing action for separating a desired target smoothly by constituting a conjugate by mixing a sample comprising a targeted biomolecule and magnetic particles, and this will be described in detail later explaining a driving method of biochip (400).

In the third region (C), 3 or more of separation channels (430) are arranged in order. In FIG. 4, 7 separation channels (430) are arranged, and referring again to FIG. 1, 4 separation channels (431, 432, 433, 434) are arranged in order. The size of separation channel (430), that is its width may be in the unit of minute micro (pm) or unit of nanometer (nm) as same as the injection channel (410), but not limited thereto, and the size may be appropriately controlled by those skilled in the art as required. In other words, in order that a conjugate in which a targeted biomolecule and magnetic particles are combined passes, the separation channel to pass may be designed in a bigger size.

On the other hand, the meaning of “arranged in order” of injection channels (410) in the first region (A) and separation channels (430) in the third region (C) means that the injection channels (410) and the separation channels (430) are arranged in order in a vertical direction on the horizontal plane to the one direction, in order to form a channel in one direction to be extended, namely in the direction from the first region (A) to the third region (C).

On the other hand, referring again to FIG. 4, a process of separating a target biomolecule by a magnetic force will be described. A sample consisting of biomaterials (30, 40, 50) comprising target biomolecules (30, 50) to be separated may be injected to some channels of injection channels (410) in the first region in which injection channels (410) are formed, and magnetic substances (10, 20) may be injected to other channels of injection channels (410). In addition, it is not specified in drawings, a liquid medium such as physiological saline (PBS, phosphate buffer saline) may be injected in other channels for smooth flow of sample and magnetic substances and smooth mixing. The magnetic substances (10, 20) may be magnetic substances in a globular form, but not limited thereto. The injection location of biomaterials (30, 40, 50) and magnetic substances (10, 20) may be arranged in consideration of direction of magnetic force (M). In other words, if the magnetic force is from the bottom up direction as FIG. 4, the magnetic substances (10, 20) are arranged lower than biomaterials (30, 40, 50), and the magnetic substances (10, 20) may be mixed in the mixing channel (420) while moving to the direction of magnetic force (M).

In the mixing channel (420), the biomaterials (30, 40, 50) and the magnetic substances (10, 20) are mixed each other and the target biomolecules (30, 50) and the magnetic substances (10, 20) are combined by immunobinding to form conjugates (60, 70), and the conjugates (60, 70) pass at least one or more of the 3 or more of separation channels (430), and the biomaterial (40) except for the target biomolecules pass the other 1 or more. In addition, a medium such as water injected in the injection channel may pass other separation channels. In other words, in the mixing channel (420), the magnetic substances (10, 20) may be mixed with the biomaterials (30, 40, 50) as moving to the direction of magnetic force (M). A probe inducing immunobinding is formed on the surface of the magnetic substances, and the target biomolecules (30, 50) to be separated by mixing in the mixing channel (420) are combined by immunobinding each other, and it can move to the direction of magnetic force (M) as same as the magnetic substances (10, 20), and the other biomaterial (40) is not affected by the magnetic force (M), and therefore it can progress in the direction of progress itself. Through this process, the target biomolecules (30, 50) can be separated from the biomaterials (30, 40, 50) injected to a sample.

In other words, in the biochip (400), the magnetic substances (10, 20), in which a probe inducing immunobinding with the biomolecule is formed on the surface is formed, may be injected to at least 1 or more of the 3 or more of injection channels (410), and the biomaterials (30, 40, 50) including the target biomolecules (30, 50) to be separated may be injected to the other 1 or more, and a delivery medium such as water may be injected to the other. It may be characterized by that in the mixing channel (420), the biomaterials (30, 40, 50) and the magnetic substances (10, 20) are mixed each other, and the target biomolecules (30, 50) and the magnetic substances (10, 20) are combined by immunobinding to form conjugates (60, 70), and the conjugates (60, 7) pass at least 1 or more of the 3 or more of separation channels (430), and the other biomaterial (40) passes the other 1 or more, and a delivery medium such as water pass the other. However, the other biomaterial (40) may comprise a small amount of the partially non-separated target materials (30, 50).

Referring again to FIG. 4, the magnetic substance may comprise a first magnetic substance (10) and a second magnetic substance which have different sizes or magnetization properties each other, and the first magnetic substance (10) and the second magnetic substance (20) may form a probe inducing immunobinding with respectively different biomolecules each other, and in the mixing channel (420), the first magnetic substance (10) and the second magnetic substance (20) and each target biomolecule (30, 50) may be combined to form a first conjugate (60) and a second conjugate (70), and in the separation channel (430), the first conjugate (60) and the second conjugate (70) may pass respectively different channels.

Since the first magnetic substance (10) and the second magnetic substance (20) have different sizes of moving magnetic force each other due to different sizes or magnetization properties each other, the level of moving to the direction for which the magnetic force (M) heads by the magnetic force (M) may be different. For example, when the first magnetic substance (10) has bigger moving magnetic force, it may be more affected by the magnetic force (M), thereby moving to the direction of magnetic force (M), and the second magnetic substance (2) moves to the direction of magnetic force (M) relatively less, and therefore the location to be injected may be different each other in the separation channel (430). In addition, since the level of moving of the first conjugate (60) and the second conjugate (70) in which the first magnetic substance (10) and the second magnetic substance (20) are combined with the target biomolecules (30, 50) is different each other, the location to be injected in the separation channel (430) may be different. On this wise, by the different sizes of moving magnetic force each other, multiple biomaterials may be separated at the same time, and thus more effective detection of target biomolecules is possible.

For the change of size of moving magnetic force of the first magnetic substance (10) and the second magnetic substance (20), the size may be changed in the magnetic substance of the same material, and materials having different magnetization each other in the same size each other may be used for preparation. In addition, it can be controlled by appropriately combining materials having different sizes each other and different magnetization each other.

On the other hand, an angle (θ) formed by connecting the virtual extended surface of the 1-1 surface (110) and the 2-1 surface (210) may be in the range of more than 0° to less than 90°. In addition, more preferably, the angel (θ) formed by connecting the virtual extended surface of the 1-1 surface (110) and the 2-1 surface (210) may be in the range of more than 0° to 50° or less. In the range, a target biomolecule can be more efficiently separated, and the range of available magnetic force can be broadened.

To explain more specifically the angle formed by the virtual extended surface, if the 1-1 surface (110) and the 2-1 surface (210) are a plane form, the part, in which the virtual surface extending the plane of the 1-1 surface (110) and the 2-1 surface (210) infinitely is connected, may have a specific angle, and the angle (θ) defined in the present invention means the above angle. On the other hand, the angle (θ) has various usage and utilization uses for separation of target biomolecule according to the change of its value, and it will be described in more detail later.

On the other hand, at least a part of the 1-1 surface (110) or the 2-1 surface (210) may include a plane or a curved surface. In other words, the 1-1 surface (110) and the 2-1 surface (210) may be consist of plane all or may be consist of curved surface all, and any one of the 1-1 surface (110) and the 2-1 surface (210) consists of plane and the other consists of curved surface, or the 1-1 surface (110) or the 2-1 surface (210) may be formed in the way of comprising the plane and curved surface respectively, etc. Like this, the separation of target biomaterial is possible in various ways by varying the magnetic force change according to the location, as the 1-1 surface (110) or the 2-1 surface (210) includes a plane or curved surface.

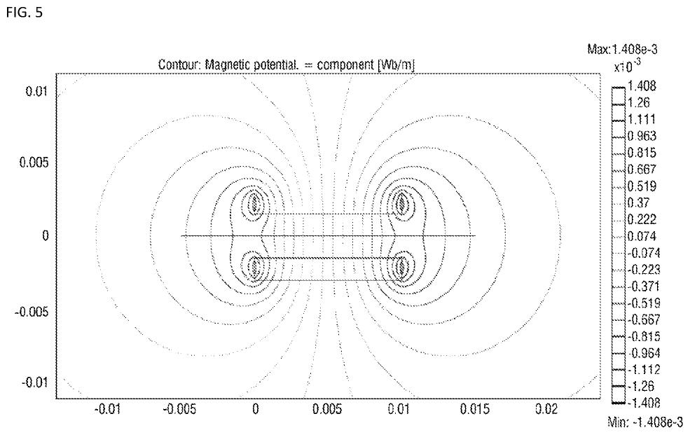

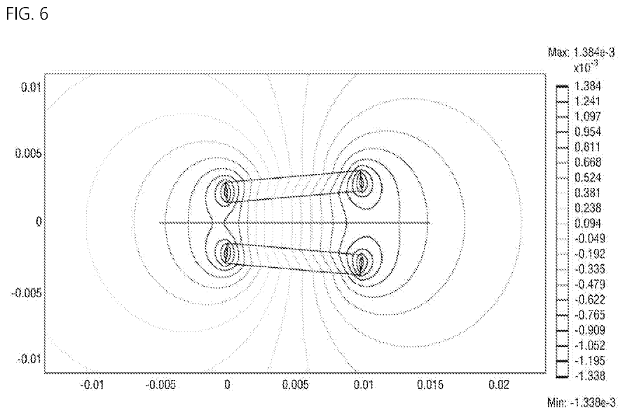

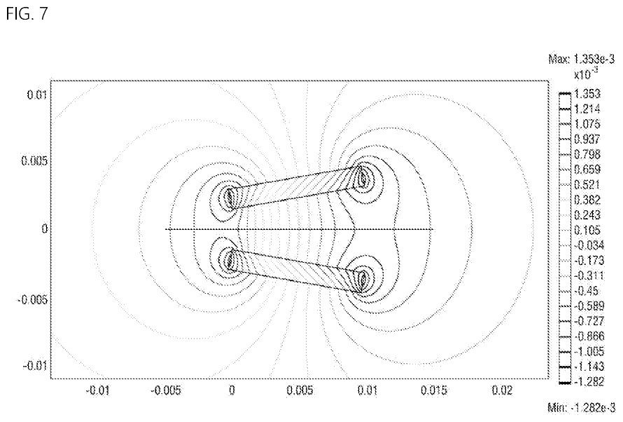

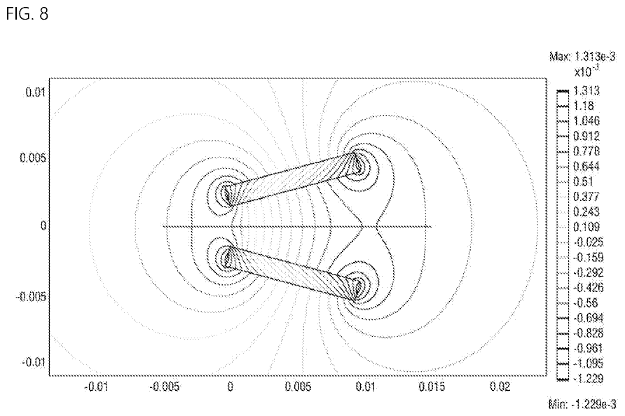

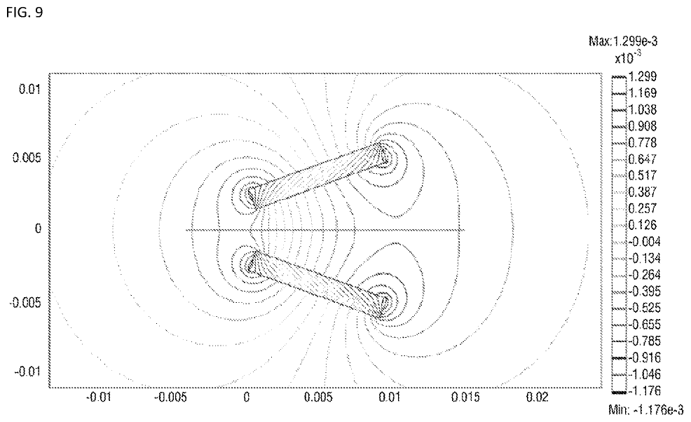

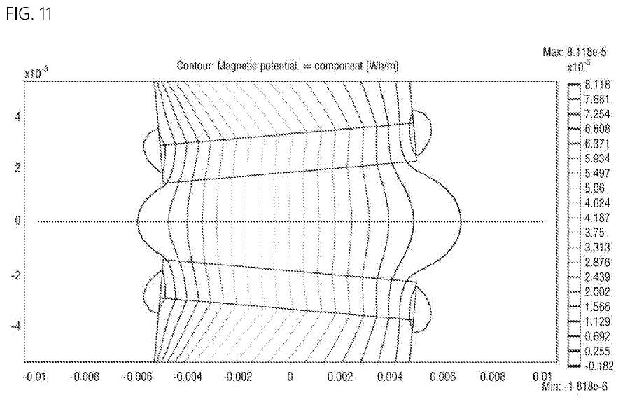

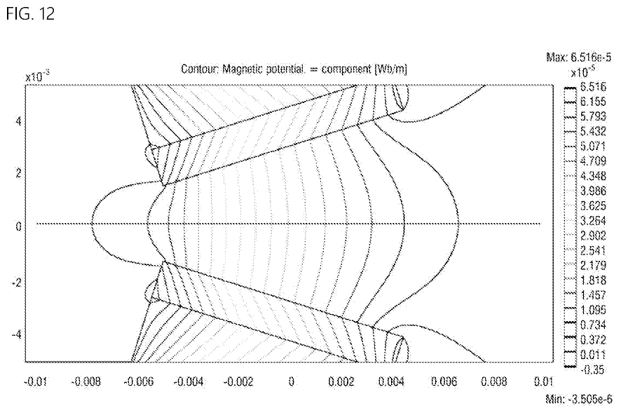

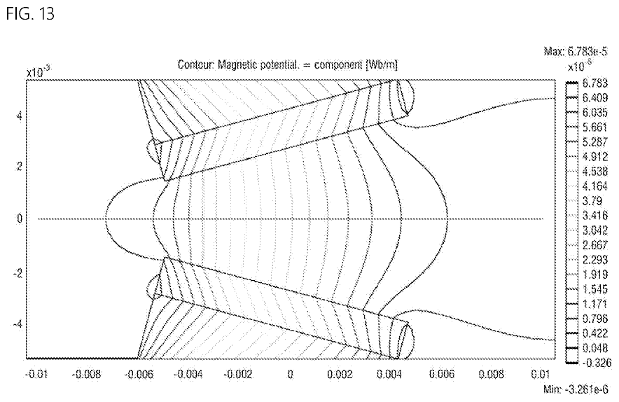

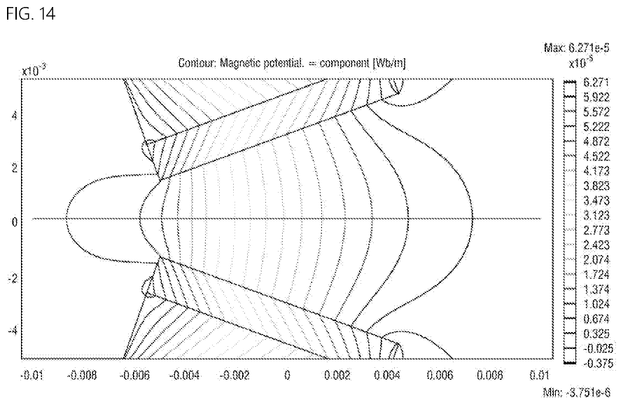

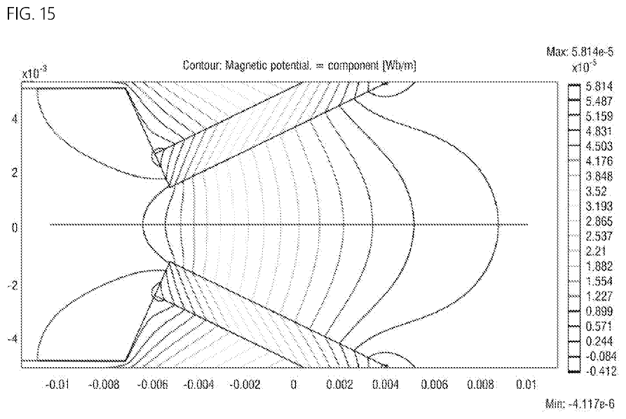

On the other hand, hereinafter, in order to compare the influence of magnetic force with and without magnetic force shield (300) by reflecting the angle (θ), the result of magnetic field interpretation will be described with reference to FIG. 5 to FIG. 9 and FIG. 10 to FIG. 15.

In FIG. 5 to FIG. 9, the result showing the magnetic field interpretation according to the simulation of comparative example in case that the angle formed by connecting the virtual extended surface of the 1-1 surface and the 2-1 surface is 0°, 10°, 20°, 30°, 40° without the magnetic force shield is shown.

As described above for the magnetic force shield (300), referring to FIG. 5 to FIG. 9, it can be seen that other magnetic forces other than magnetic force effects by the 1 pole (N) and the 2 pole (S) affect the gradient of magnetic force between the 1-1 surface and the 2-1 surface. As above, the meaning of affecting the gradient of magnetic force between the 1-1 surface and the 2-1 surface much means that it is difficult for users using the magnetophoresis biochip to predict the moving direction, moving strength, etc. of magnetic substance by the magnetic force, and means that the controllable range for users become very narrow.

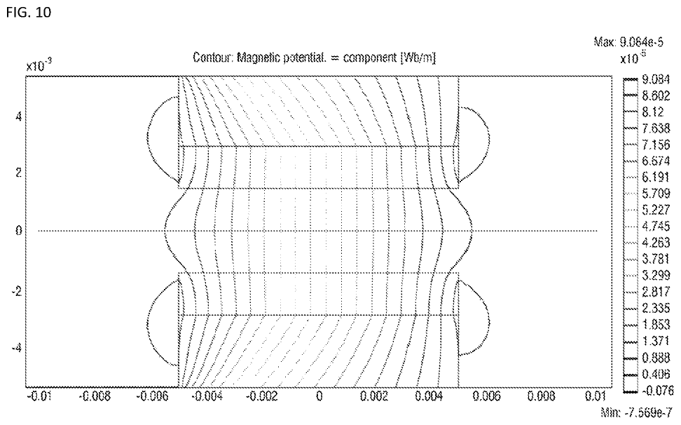

Different from the above FIG. 5 to FIG. 9, in FIG. 10 to FIG. 15, the result showing the magnetic field interpretation according to the simulation of examples of the present invention in case that the angle formed by connecting the virtual extended surface of the 1-1 surface and the 2-1 surface is 0°, 10°, 20°, 30°, 40°, 50° is shown.

As FIG. 10 to FIG. 15, when the magnetic force shield is equipped according to the examples of the present invention, it can be seen that as many parts have uniform difference of strength of magnetic forces between the 1-1 surface and the 2-1 surface, it is easy to predict the level of change of magnetic force, and the controllable range for users using the magnetophoresis biochip becomes relatively much broadened than that without the magnetic force shield (300). Thus, it can be used for utilization of biochip in broader section, and it is easy to control to the desirable strength of magnetic force of users. This is because the biochip is affected only by the control of magnetic force between the 1 pole and the 2 pole, and this is because other poles except for the 1 pole and the 2 pole are excluded by the magnetic force shield.

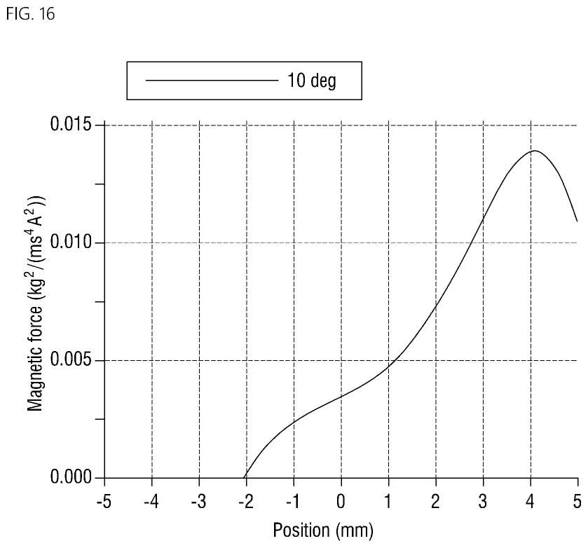

FIG. 16 to FIG. 20 show the result of magnetic force shown by the simulation by varying the angle formed by connecting the virtual extended surface of the 1-1 surface and the 2-1 surface of the magnetophoresis biochip according to the example of the present invention. Hereinafter, a process of separating a target biomolecule of biochip using a magnetic force gradient in a specific angle will be described. On the other hand, before explaining FIG. 16 to FIG. 20, to explain with reference to FIG. 10 to FIG. 15, it can be seen that the strength of magnetic force becomes bigger from the right to the left, and that an object having a magnetic property moves from the right to the left, and it will be appreciated that an object having a magnetic property moves in the same way also in FIG. 16 to FIG. 20. In addition, it will be appreciated that the distance of the 1 surface and the 2 surface becomes closer from the right to the left.

Then, referring to FIG. 16, when the angle formed by connecting the virtual extended surface of the 1-1 surface and the 2-1 surface is 10°, it can be seen that the strength of magnetic force is gradually decreased. This means that the closer the distance of the 1 surface and the 2 surface is, the gradually lower the strength of magnetic force is, thereby gradually reducing the power of attracting the magnetic substance. Thus, it can be seen that as the magnetic substance moves from the right to the left, the magnetic substance is rapidly drawn by the magnetic force at the beginning and gradually moves slowly as going to the left or by the power of small magnetic force. In a magnetophoresis microfluid chip using a general single magnet, the magnetic force is continuously increased in the direction of moving, and therefore when the magnetic substance is combined to the target biomolecule and then moved to the left direction by the gradient of magnetic force, the power by the magnetic force becomes bigger than the flow power by the flow of fluid, and ultimately it cannot progress to the separation channel, it may be accumulated in a specific location. However, as the magnetic force becomes weaker as going to the left, when having the gradient of magnetic force as FIG. 16, the accumulation as above can be prevented, and the magnetic substance may be immobilized in the location where the magnetic force is 0, and thereby the fluid in the location may be injected to the corresponding separation channel.

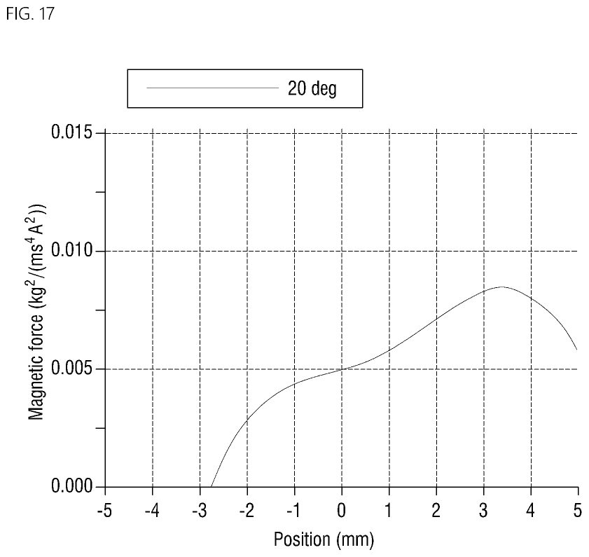

Referring to FIG. 17, it can be seen that when the angle formed by connecting the virtual extended surface of the 1-1 surface and the 2-1 surface is 20°, the strength of magnetic force becomes gradually decreased from the right to the left, but the width of decrease is reduced in the middle part compared to the angle of 10°. Using this, the location where the magnetic substance and the target biomolecule are combined may be located in the middle part where the width of decrease of magnetic force is reduced. In other words, the power of attracting by the magnetic force may be more needed when the target biomolecule and the magnetic substance are combined, and this may be utilized in such a middle part. In addition, as FIG. 16, it may be prevented that the conjugate is accumulated in a specific location that is the end of biochip, by undermining the power of magnetic force when going to further to the left from the middle part.

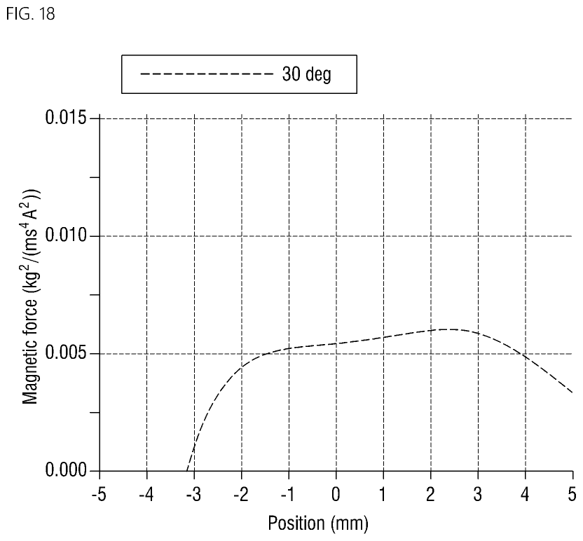

Referring to FIG. 18, it can be seen that when the angle formed by connecting the virtual extended surface of the 1-1 surface and the 2-1 surface is 30°, as going from the right to the left, the power of magnetic force becomes bigger in the part located in the right and the power of magnetic force is gradually reduced in the middle part, and the magnetic force is rapidly reduced in the left part. Using this, the mixing time of the magnetic substance and the target biomolecule may be extended by alleviating the speed of magnetic substance in the middle part, and it may be prevented that the conjugate is accumulated in a specific location that is the end of biochip by weakening the power of magnetic force when going to further to the left in the middle part. In addition, the total separation time of target biomolecule may be decreased by gradually increasing the power of magnetic force in the right part.

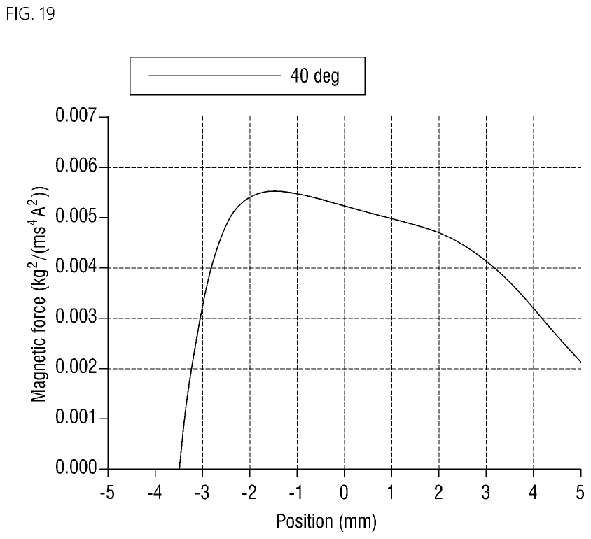

Referring to FIG. 19, it can be seen that when the angle formed by connecting the virtual extended surface of the 1-1 surface and the 2-1 surface is 40°, the strength of magnetic force is increased as going from the right to the left, and the width of increase is reduced at a certain level in the middle part, and it is reduced again when going to further to the left. Using this, the binding time of target biomolecule may be extended by making the magnetic substance move fast at the beginning and making it move more slowly than the beginning in the middle part, and it may be prevented that the conjugate is accumulated in a specific location that is the end of biochip by weakening the power of magnetic force when going to the left from the middle part. The availability of FIG. 19 is that the separation is completed in a shorter time compared to FIG. 18, and those skilled in the art can appropriately select and use it according to characteristics, size, etc. of target biomolecule to be separated.

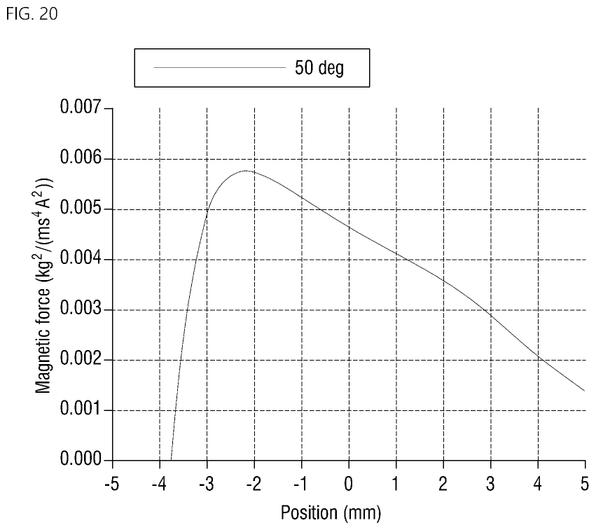

On the other than, referring to FIG. 20, it can be seen that when the angle formed by connecting the virtual extended surface of the 1-1 surface and the 2-1 surface is 50°, the strength of magnetic force is uniformly increased from the right to the left by the middle part, and it is rapidly reduced in the left end. Using this, the magnetic substance may be injected in the right, and the speed may be gradually faster as passing the middle part. When the size of target biomolecule is big or its weight is heavy, the power more than the specific magnetic force may be required to move the conjugate, and for this, the gradient of magnetic force such as FIG. 20 may be used. As above, when going to the left end, the strength fo magnetic force is gradually reduced, and thereby it may be prevented that the conjugate is accumulated in a specific location of biochip.

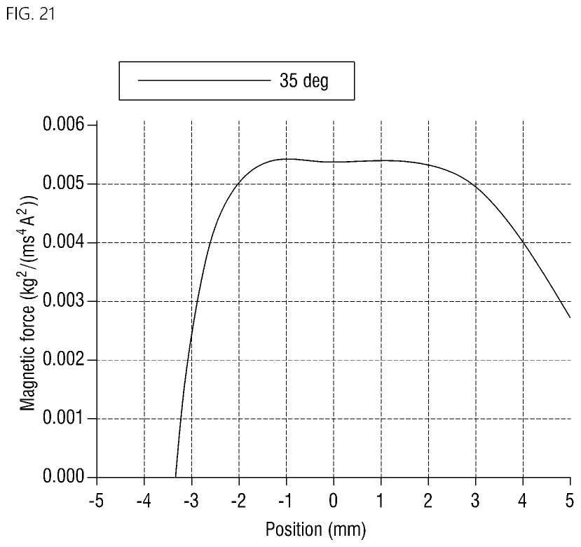

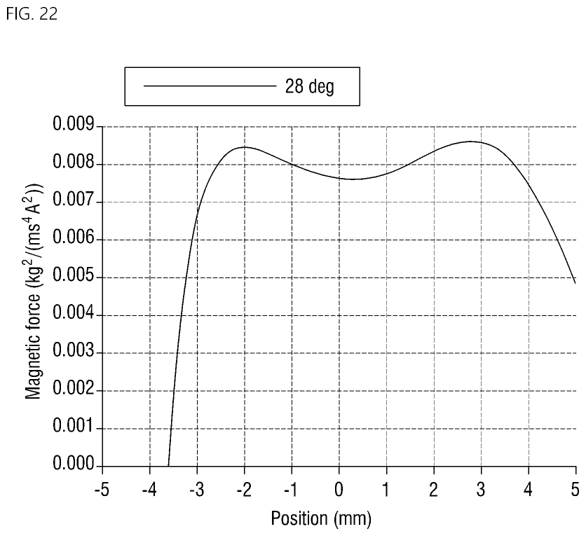

In FIG. 21 and FIG. 22, the result of magnetic force shown by the simulation when the angles formed by connecting the virtual extended surface of the 1-1 surface and the 2-1 surface are 35° and 28° respectively, when the closet distance of the 1-1 surface and the 2-2 surface of the magnetophoresis biochip according to the example of the present invention is shown. In addition, in FIG. 21 and FIG. 22, there is the result when the width of the 1-1 surface and the 2-1 surface is 10 mm.

Referring to FIG. 21 and FIG. 22, the length range forming the effective magnetic force in contrast to the distance spaced apart of the 1-1 surface and the 2-1 surface may form the effective magnetic force in the width range of approximately 30% to 50% compared to the width of the 1-1 surface and the 2-1 surface. In other words, when the width of the 1-1 surface and the 2-1 surface is 10 mm, the effective magnetic force capable of using the biochip may be formed in the range of 3.0 mm to 5.0 mm. Referring to FIG. 21, the range of effective magnetic force may be within the range of −1.2 mm to 1.8 mm or the range of −2.0 mm to 3.0 mm transversely, and referring to FIG. 22, the range of effective magnetic force may be in the range of −1.0 mm to 1.5 mm or the range of −2.7 mm to 3.8 mm. However, it is not limited thereto.

On the other hand, the ratio of the width of the 1-1 surface and the 2-2 surface and the closest distance spaced apart between the 1-1 surface and the 2-2 surface may be in the range of 30:1 to 1:1 or in the range of 20:1 to 5:1. In the above range, the effective magnetic field gradient between the 1-1 surface and the 2-2 surface can be obtained more effectively.

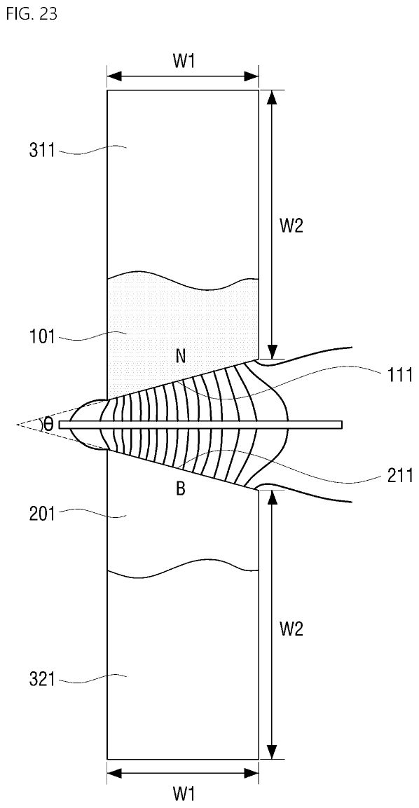

In FIG. 23, a sectional view schematically showing the magnetophoresis biochip of other example of the present invention is shown. Referring to FIG. 23, it is characterized by that the first magnetic force shield (311) is formed by being extended in the direction opposite to the 2-1 surface (210) in the 1-1 surface (110) by extending the 1-1 pole of the magnetic force providing unit, and the second magnetic force shield (321) is formed by that the 2-1 pole of the magnetic force providing unit is extended in the direction opposite to the 1-1 surface (110) in the 2-1 surface (210), and the first magnetic force shield (311) has the ratio of width (W1) of the 1-1 surface (110) and the extended height (W2) of the 1-1 pole of 1:3 or more to 1:100 or less, and the second magnetic force shield (321) has the ratio of width (W1) of the 2-1 surface and the extended height (W2) of the 2-1 pole of 1:3 or more to 1:100 or less. By making the height (W2) part higher like the ratio of the width (W1) and the height (W2), it may be prevented that the 1-2 pole and the 2-2 pole affect the gradient of magnetic force between the 1-1 surface (110) and the 2-1 surface (210), and the ratio of width (W1) and height (W2) may be in the range of 1:3 or more to 1:5 or less, regarding the convenience of use and size of equipment configuration. As a non-limitative example, the widths of the 1-1 surface and the 2-1 surface may be same, and the heights (W2) of the first magnetic force shield (311) and the second magnetic force shield (321) may be substantially same.

Even if the magnetic force except for the 1 pole (N) and the 2 pole (S) is completely blocked by the above ration, it may be blocked that other magnetic forces affect the magnetic force between the 1-1 surface (111) and the 2-1 surface (211).

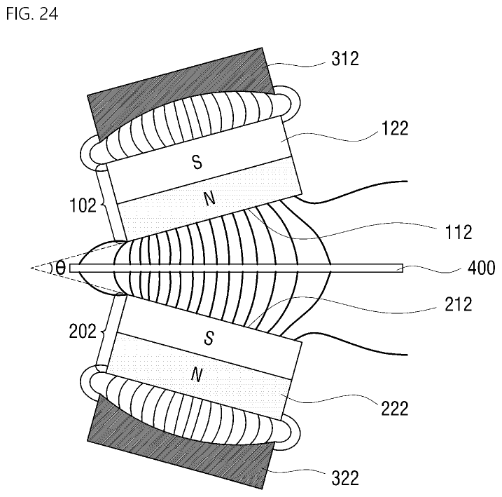

In FIG. 24, a sectional view schematically showing the magnetophoresis biochip of other example of the present invention is shown. Referring to FIG. 24, the first magnetic force shield (312) may be arranged as spaced apart from the 1-2 surface which is the opposite surface to the 1-1 surface (112) facing the 2-1 surface (212) and be formed as the same pole with the 1-1 pole (N), and the second magnetic force shield (322) may be arranged as spaced apart from the 2-2 surface which is the opposite surface to the 2-1 surface (212) facing the 1-1 surface (112) and be formed as the same pole with the 2-1 pole (S). More specifically, in case of permanent magnet, as FIG. 24, the 1-2 pole formed in the opposite surface of the 1-1 pole (N) may be formed as the same pole (S) with the 2-1 pole (S), and when arranging the first magnetic force shield (312) consisting of the same pole with the 1-1 pole (N) as spaced apart in order to offset this pole, it may be prevented that the magnetic force in the opposite surface affects the gradient of magnetic force between the 1-1 surface (110) and the 2-1 surface (210). The second magnetic force shield (322) may be perform the function of magnetic force shield on the same principle as the first magnetic force shield (312).

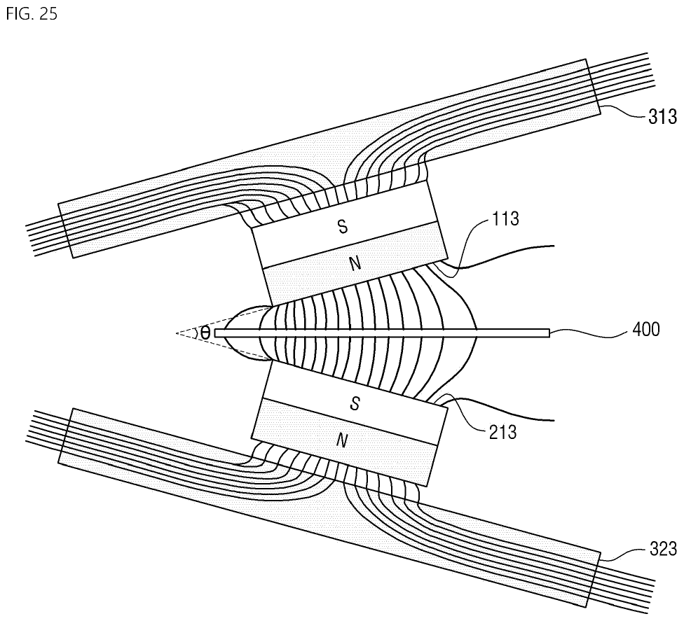

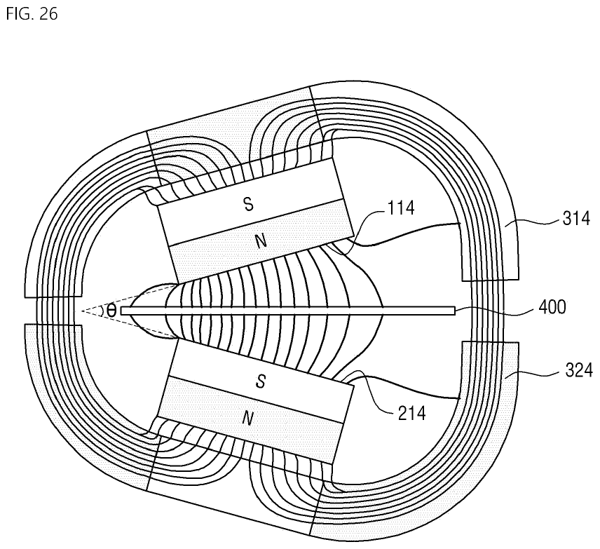

In FIG. 25 and FIG. 26, sectional views schematically showing the magnetophoresis biochip of other example of the present invention are shown. At first, referring to FIG. 25, it is characterized by that the first magnetic force shield (313) makes the magnetic force by the 1-2 pole not overlapped with the magnetic field formed between the 1-1 surface (113) and the 2-1 surface (213), by inducing the magnetic force of the 1-2 pole in a specific direction as being in contact with or spaced apart from the 1-2 surface which is the opposite surface of the 1-1 surface (113) facing the 2-1 surface (213), and the second magnetic force shield (323) makes the magnetic force by the 2-2 pole not overlapped with the magnetic field formed between the 1-1 surface (113) and the 2-1 surface (213), by inducing the magnetic force of the 2-2 pole in a specific direction as being in contact with or spaced apart from the 2-2 surface which is the opposite surface of the 2-1 surface (213) facing the 1-1 surface (113).

In other words, the first magnetic force shield (313) and the second magnetic force shield (323) induce the magnetic force occurring in the 1-2 pole and the 2-2 pole to other region except for the region between the 1 surface (113) and the 2 surface (213), thereby making not affecting the gradient of magnetic field between the 1 surface (113) and the 2 surface (213). In addition, the effect of magnetic force which can be penetrated from the outside except for the region shown in the drawings may be not affecting the region between the 1 surface (113) and the 2 surface (213). For this, as FIG. 25, the first magnetic force shield (313) and the second magnetic force shield (323) may be formed to cover magnetic force providing unit all and to deviate to the outside of it.

Meanwhile, in FIG. 26, examples of the first magnetic force shield (314) and the second magnetic force shield (324) in different forms from the FIG. 25 are shown. Referring to FIG. 26, the first magnetic force shield (314) and the second magnetic force shield (324) may be a form of facing each other as a bent shape, and may have a circular shape on the whole. In addition, the first magnetic force shield (314) and the second magnetic force shield (324) may induce an external magnetic force or magnetic force other than the magnetic field between a 1 surface (114) and a 2 surface (214) to the outside of the 1 surface (114) and the 2 surface (214) more easily, by combining magnetic poles respectively different each other in order.

On the other hand, the magnetic force shield described in FIG. 25 and FIG. 26 may comprise a nickel-iron soft magnetic alloy material as a non-limitative example, but not limited thereto.

The examples of the present invention are described with reference to accompanying drawings above, but the present invention is not limited to the examples, and it may be prepared in various forms different each other, and those skilled in the art may appreciate that it may be implemented in other specific forms without modifying technical spirits or essential features of the present invention. Therefore, the examples described above should be appreciated as illustrative and not limitative in all aspects.

What is claimed is:

1. A magnetophoresis biochip comprising a magnetic force providing unit including a 1-1 surface having magnetic force of a 1-1 pole, and a 2-1 surface which is spaced apart from the 1-1 surface and faces each other and has magnetic force of a 2-1 pole that is opposite to the 1-1 pole;
a magnetic force shield including a first magnetic force shield blocking magnetic force of a 1-2 pole of opposite side of surface facing the 2-1 surface in the 1-1 surface, and a second magnetic force shield blocking magnetic force of a 2-2 pole of opposite side of surface facing the 1-1 surface in the 2-1 surface; and
a biochip including 3 or more injection channels, a mixing channel and 3 or more separation channels, which are located between the 1-1 surface and the 2-1 surface and extended in one direction and arranged in sequence,
wherein an angle formed by connecting a virtual extended surface of the 1-1 surface and the 2-1 surface is in a range of more than 0° to less than 90°.

2. The magnetophoresis biochip of claim 1, wherein the first magnetic force shield is formed by that the 1-1 pole of the magnetic force providing unit is extended in a direction opposite to the 2-1 surface in the 1-1 surface, and the second magnetic force shield is formed by that the 2-1 pole of the magnetic force providing unit is extended in a direction opposite to the 1-1 surface in the 2-1 surface, and the first magnetic force shield has a ratio of a width of the 1-1 surface and an extended height of the 1-1 pole of 1:3 or more to 1:100 or less, and the second magnetic force shield has a ratio of a width of the 2-1 surface and an extended height of the 2-1 pole of 1:3 or more to 1:100 or less.

3. The magnetophoresis biochip of claim 1, wherein the magnetic force shield is formed by that the 1-1 pole of the magnetic force providing unit is extended, and it is formed by that the 2-1 pole is extended, and an end of extended part of the 1-1 pole and an end of extended part of the 2-1 pole are in contact with each other.

4. The magnetophoresis biochip of claim 1, wherein the first magnetic force shield is arranged as spaced apart from the 1-2 surface which is an opposite surface of the 1-1 surface facing the 2-1 surface, and is formed with the same pole as the 1-1 pole,
and the second magnetic force shield is arranged as spaced apart from the 2-2 surface which is an opposite surface of the 2-1 surface facing the 1-1 surface, and is formed with the same pole as the 2-1 pole.

5. The magnetophoresis biochip of claim 1, wherein the first magnetic force shield prevents the magnetic force by the 1-2 pole from overlapping with a magnetic field formed between the 1-1 surface and the 2-1 surface, by being contact with or spaced apart from the 1-2 surface which is an opposite surface to the 1-1 surface facing the 2-1 surface to induce the magnetic force of the 1-2 pole in a specific direction, and the second magnetic force shield prevents the magnetic force by the 2-2 pole from overlapping with the magnetic field formed between the 1-1 surface and the 2-1 surface, by being contact with or spaced apart from the 2-2 surface which is an opposite surface to the 2-1 surface facing the 1-1 surface to induce the magnetic force of the 2-2 pole in a specific direction.

6. The magnetophoresis biochip of claim 1, wherein an angle formed by connecting a virtual extended surface of the 1-1 surface and the 2-1 surface is in the range of more than 0° to 50° or less.

7. The magnetophoresis biochip of claim 1, wherein at least a part of the 1-1 surface or the 2-1 surface includes a plane or a curved surface.

8. The magnetophoresis biochip of claim 1, wherein a ratio of a length of width of the 1-1 surface or the 2-1 surface and a closest distance between the 1-1 surface and the 2-1 surface is in the range of 30:1 to 1:1.

9. The magnetophoresis biochip of claim 1, wherein in the biochip, a magnetic substance, in which a probe for inducing an immunobinding with a biomolecule on a surface is formed, is injected to at least one or more of the 3 or more injection channels, and a biomaterial containing a target biomolecule to be separated is injected to the other 1 or more,
and in the mixing channel, the biomaterial and the magnetic substance are mixed with each other, and the target biomolecule and the magnetic substance are combined by immunobinding to form a conjugate,
and the conjugate is passing through at least one or more of the 3 or more of separation channels, and the biomaterial is passing through the other 1 or more.

10. The magnetophoresis biochip of claim 9, wherein the biochip comprises 4 or more of injection channels, a mixing channel and 4 or more of separation channels,
and the magnetic substance comprises a first magnetic substance and a second magnetic substance which have a different size from each other or characteristic of magnetization, and the first magnetic substance and the second magnetic substance form a probe for inducing an immunobinding with a different biomolecule each other respectively,
and in the mixing channel, the first magnetic substance and the second magnetic substance are combined with each target biomolecule to form a first conjugate and a second conjugate,
and in the separation channel, the first conjugate and the second conjugate pass through a respectively different channel.
